(19) United States
(12) Patent Application Publication
(10) Pub. No.: US 2009/0214619 A1
(43) Pub. Date: Aug. 27, 2009

(54) IMPLANT FOR INTRAOCULAR DRUG DELIVERY

(75) Inventors: Andreas REIFF, San Marino, CA (US); Scott HAMPTON, Cumming, GA (US); Richard PAYNE, Winston-Salem, NC (US)

Correspondence Address: WILSON SONSINI GOODRICH & ROSATI, 650 PAGE MILL ROAD, PALO ALTO, CA, 94304-1050, US

(73) Assignee: THERAKINE LTD., Cumming, GA (US)

(21) Appl. No.: 12/406,876

(22) Filed: Mar. 18, 2009

Related U.S. Application Data
(62) Division of application No. 11/280,377, filed on Nov. 16, 2005.
(60) Provisional application No. 60/630,751, filed on Nov. 24, 2004.

(51) Int. Cl. A61F 2/14; A61K 39/395; A61K 38/20

(52) U.S. Cl. 424/427; 424/133.1; 424/85.2

(57) ABSTRACT

An implant for intraocular drug delivery for the treatment of inflammatory or degenerative diseases. In one embodiment, the implant includes a body portion having a first end portion and a second, opposite end portion and defining a cavity with a first opening at the first end portion, and a second, opposite opening at the second end portion, and a solid material received in the cavity, wherein the solid material comprises a depot material and an effective amount of at least one therapeutic compound or agent. When the implant is implanted in an eye of a living subject, the effective amount of at least one therapeutic compound or agent is released to the environment of the implant through at least one of the first opening and the second, opposite opening over an extended period of time.

### CROSS-REFERENCE TO RELATED PATENT APPLICATION

This application claims the benefit, pursuant to 35 U.S.C. §119(e), of U.S. provisional patent application Ser. No. 60/630,751, filed Nov. 24, 2004, entitled “EYE IMPLANT WITH MEDICINE RELEASE,” by Scott M. Hampton and Andreas Reiff, which is incorporated herein by reference in its entirety.

Some references, if any, which may include patents, patent applications and various publications, are cited and discussed in the description of this invention. The citation and/or discussion of such references is provided merely to clarify the description of the present invention and is not an admission that any such reference is “prior art” to the invention described herein. All references, if any, cited and discussed in this specification are incorporated herein by reference in their entireties and to the same extent as if each reference individually incorporated by reference. In terms of notation, hereinafter, “[n]” represents the nth reference cited in the reference list. For example, [10] represents the 10th reference cited in the reference list, namely, Franks W A. Limb G A. Stanford M R. Ogilvie J. Wolstencroft R A. Chignell A H. Dumonde D C., Cytokines in human intraocular inflammation, Current Eye Research. 11 Suppl: 187-91, 1992.

### FIELD OF THE INVENTION

The present invention is generally related to an ocular implant, and more particularly, is related to an implant having at least one compound or agent releasable for the treatment of intraocular diseases therein.

### BACKGROUND OF THE INVENTION

Many chronic disorders of the eye may and can cause long-term damage including vision loss or blindness. Two main categories of diseases may be differentiated: the non-infectious chronic inflammatory eye diseases and the degenerative vasculopathies such as age related macular degeneration or diabetic retinopathy. Recent research suggests that inflammatory mechanisms contribute to degenerative diseases of the eye [19, 20, 21, 22, 23], so the categories may be more descriptive than casual and may have overlapping features.

In the first category, inflammatory eye diseases, the barrier that shields the eye from an invasion of auto aggressive white blood cells is disrupted by an autoimmune process allowing “eye foreign” white blood cells to invade the eye and attack its inner layers. The term uveitis refers to intraocular inflammations, which accounts for approximately 50 different entities with either infectious or autoimmune origin. The intraocular inflammation generally originates from the middle layer of an eye of a living subject, called a uvea. The uveal tract of the eye includes an iris, a ciliary body, and a choroid. Inflammation of the overlying retina, called retinitis, or of the optic nerve, called optic neuritis, may occur with or without accompanying uveitis. Primary uveitis (“idiopathic”) is referred to the intraocular inflammation of unknown cause (roughly 40% of cases seen in tertiary referral centers). Secondary uveitis (all cases with some explanation for the uveitis) accounts for inflammatory ocular conditions that are either associated with a systemic disease (e.g. ankylosing spondylitis or sarcoidosis) of known infectious cause (e.g. toxoplasmosis or CMV-retinitis) or defined as ocular syndromes (e.g. Fuchs uveitis syndrome, Birdshot syndrome or serpiginous choroiditis). Masquerade syndromes, like intraocular lymphoma, are different from primary or secondary uveitis.

The etiology and pathogenesis of uveitis is not yet fully understood. Uveitis can be caused by infections, malignancy, exposure to toxins and autoimmune disorders. Disturbances of immune mechanisms have long been suspected of playing a central role in intraocular inflammation. In the majority of cases of endogenous uveitis in which no link with an infectious agent can be identified, autoimmunity has been believed as the cause.

Clinic data collected from animals suggest that susceptibility to autoimmune uveitis is caused by a predominant Th1 response of autoreactive T cells against retinal antigens. Th1 cells mainly produce cytokines such as INF gamma, IL2, 12, 18 while TNF is mainly associated with cell-mediated autoimmunity. The significantly elevated ocular and systemic levels of IL-1 beta and TNF suggest that there is not only a localized ocular response but a systemic response as well. The presence of IL-1 beta and TNF may play a role in the pathogenesis of ocular inflammation once the blood ocular barrier has been breached and ocular antigens have been exposed to the systemic immune system. Particularly, IL-6 and IL-1 may act as local amplification signals in pathological processes associated with a chronic eye inflammation. Additionally, other proinflammatory cytokines such as IL2, IL4, IL6, IL8, IL12, IL15, IL17, IL18 and chemokines such as Matrix Metallo Proteinases (MMPs) play an important role in the chronic inflammation of the eye.

The incidence of uveitis appears to be increasing over the last decade and is approximately 52.4/100,000 person-years with a period prevalence of 115.3/100,000 persons. Uveitis afflicts approximately 420,000 Americans annually. The rate of the incidence and prevalence of uveitis is lowest in pediatric age groups, increases with age and is highest in patients 65 years old and older.

Ocular complications of uveitis produce profound and irreversible loss of vision, especially when such ocular complications are unrecognized and/or treated improperly. Some of the most frequent complications include cataract, glaucoma, retinal detachment, cystoid macular edema, neovascularization of the retina, optic nerve and iris.

The long-term outcome of uveitis in adults is unknown because no prospective studies are available. In the pediatric population with autoimmune conditions (such as juvenile rheumatoid arthritis), the risk of permanent blindness after 5 years has remained unchanged at about 10%, despite aggressive treatment with topical steroids and systemic immunosuppressive therapy. About 30% have significant loss of vision, requiring lifelong assistance. Because uveitis causes pain and light sensitivity, the impact on quality of life is much more severe than the figures above indicate, even for “mild” cases.

In the second category of chronic eye diseases, degenerative vasculopathies, age related or metabolic factors cause blood vessels to obliterate and no longer supply vital parts of the eye with blood. As a result, the eye rapidly starts to form new blood vessels around the occluded old vessel in order to compensate for the lack of blood supply. Unfortunately these repair mechanisms are frequently insufficient and the newly formed blood vessels often burst resulting into bleeding into the eye and detachment of the retina.

The most important diseases in the degenerative category include age related macula degeneration and diabetic retinopathy, as well as cystoid macular edema.

Macular degeneration is the most common cause of blindness in the senior population of the developed world. In macular degeneration, the light-sensing cells of the macula malfunction and cease to work over time. Macular degeneration occurs most often in people over 60 years old, in which case it is called Age Related Macular Degeneration (AMD or ARMD) but can occur at all ages including children. The most common early sign of AMD is blurred vision, straight lines appearing wavy, and finally leading to loss of visual acuity and color sensitivity. The macula is the part of the retina that provides central vision, and as it degenerates it can lead to partial or complete loss of vision. About 85-90% of AMD cases are the dry, or atrophic, form, in which yellowish spots of fatty deposits called drusen appear on the macula. The rest of AMD cases are the wet form, so called because of leakage into the retina from newly forming blood vessels in the choroid, a part of the eye behind the retina. Normally, blood vessels in the choroid bring nutrients to, and carry waste products away from, the retina. Sometimes the fine blood vessels in the choroid underlying the macula begin to proliferate, a process called choroidal neovascularization, or CNV. The cause is unknown. When those blood vessels proliferate, they leak, and cells in the macula may be damaged and may die. Laser photocoagulation is a technique used by ophthalmic surgeons to treat leakage from submacular neovascularizations. Unfortunately only about half of patients with wet AMD are candidates for laser photocoagulation and laser photocoagulation is only effective about half the time it is done as a treatment for wet macular degeneration. When effective, the benefit lasts on the average about one year.

Diabetic retinopathy is the leading cause of acquired blindness among Americans under the age of 65. Diabetic retinopathy may occur at any point in time after the onset of diabetes. Blood vessels damaged from diabetic retinopathy can cause vision loss in two ways: Fragile and abnormal blood vessels can develop and leak blood into the center of the eye, blurring vision. This is proliferative retinopathy and is the fourth and most advanced stage of the disease. Fluid can leak into the center of the macula, the part of the eye where sharp, straight-ahead vision occurs. The fluid makes the macula swell, blurring vision. This condition is called macular edema. It can occur at any stage of diabetic retinopathy, although it is more likely to occur as the disease progresses. About half of the people with proliferative retinopathy also have macular edema.

Findings in the retina include dot and blot hemorrhages (tiny hemorrhages in the retina itself), microaneurysms (out-pouchings of capillaries), and exudates (retinal deposits occurring as a result of leaky vessels). The development of this condition in type I (juvenile-onset) diabetics is rarely present prior to three or four years following the onset of diabetes. In type II (adult-onset) diabetics, background diabetic retinopathy may be present at the time of diagnosis of the condition. The great majority of this blindness can be prevented with proper examination and treatment by ophthalmologists. Unfortunately, patients who are not properly referred for evaluation and management or those who, for any reason, fail to get proper care from an ophthalmologist, are at the greatest risk of vision loss.

Various treatment options have been developed for patients who are affected by these 2 categories of disorders.

In case of the inflammatory eye diseases, the treatments of noninfectious and/or autoimmune uveitis include administering topical steroid eyedrops and/or corticosteroids, combined with antimicrobials and cycloplegic drops. Even though most patients will have a mild form of uveitis, the disease can linger for months (many cases continue for years), and residual damage to the iris or the lens is not uncommon. Glaucoma (increased pressure in the eye) is an additional side effect of steroid eyedrops and can further limit the patient's vision. For certain cases, it may require injection of steroids into the tissue around the eye. If this is not effective, corticosteroids can be given orally, with well known side effects such as weight gain (including fat deposits developing on the face) increased risk of infections, osteoporosis, weakness, diabetes, slow wound healing with easy bruising, acne, salt retention, and hypertension. Additional risks in the eye include cataract and glaucoma.

Clinical research has shown that the use of antibodies designed to modulate elements of the immune system lead to positive outcomes in inflammatory and degenerative conditions of the eye. However, the antibody compounds must be administered systemically either by intravenous (IV) or sub-cutaneous injection. The problem with this systemic application is the risk of systemic infections, reactivation of tuberculosis and demyelination in the brain in patients with multiple sclerosis. Furthermore, since the eye is a well-shielded organ with natural barriers to the blood, treatments with antibodies require much higher doses than those requires in rheumatoid arthritis. Thus the cost of such a treatment can be prohibitively expensive.

In the case of inflammatory eye diseases, treatment is facilitated by using anti-cytokines or anti-chemokines that modulate chronic inflammatory eye disease, and a number of such drugs are being used systemically with good success. However the systemic use, such as an intravenous injection, is expensive, and is associated with side effects and not always effective. By giving these drugs directly into the eye through the device(s) and method(s) according to several embodiments of the present invention, systemic side effects can be avoided and better local control of the inflammation can be achieved. In addition the patients' immune system remains substantially unchanged since the present invention allows the modulation of local inflammation only.

For the patients with degenerative vasculopathies, among other unique features, the present invention allows direct drug delivery into the eye but instead of using anti-cytokines or anti-chemokines, protein inhibitors, so called MAP-Kinase inhibitors, will be used to precisely block intracellular signals that would lead to the formation of new blood vessels. The protein inhibitors are delivered directly into the eye over an extended time period. This in turn can prevent catastrophic bleeding from or into the eye and avoid costly laser surgeries to reattach the retina. These drugs have already been successfully used in the treatment of solid tumors where they prevent the formation of new blood vessels thereby shutting off the blood supply to the growing tumor leading to its death. Inflammation is implicated as a contributing factor in degenerative eye diseases, such as macular degeneration, and effective treatment of these diseases may require the use of multiple agents to modulate inflammation and new vessel formation.

The intracellular signal transduction pathways involved in inflammation and cell transformation and their relationship to autoimmune diseases are only beginning to be explored. The identification of enzymes involved in signaling from the plasma membrane to the nucleus in lymphocytes and the cells involved in autoimmune diseases will likely contribute significantly to future understanding of mechanisms responsible for lymphocyte differentiation and for the discrimination of self from non-self in developing and mature cells.

Chemical manipulations of the enzymes involved in these pathways known as selective kinases or downstream transcription factors provide a unique opportunity for novel therapeutic interventions. It is feasible that inhibition of specific signal transduction or transcription factor targets might interrupt the perpetuation mechanisms involved in many autoimmune diseases. The blockade of the appropriate pathway could provide an opportunity to reestablish homeostasis by inhibition of cellular responses, such as lymphokine gene expression and cellular release of proinflammatory cytokines such as TNF and others.

Despite the differences in the antigens that they recognize and in the effector functions they carry out, B and T lymphocytes utilize remarkably similar signal transduction components to initiate responses. Even though the signaling pathways are highly diverse, they display an extraordinary degree of specificity for a given transcription factor or transcription factor family. A number of transcription factor families, including those for activator protein 1 (AP-1)/activating transcription factor 2 (ATF2), nuclear factor [kappa] B (NF-[kappa] B), nuclear factor of activated T cells (NF-AT), signal transducer and activator of transcription (STAT), p53, and nuclear hormone receptors, have been implicated as critical regulators of gene expression in the setting of inflammation

In animal models of uveitis such as endotoxin-induced uveitis (EIU), a signaling pathway known as the extracellular signal-regulated kinase (ERK) pathway plays an important role in the inflammation of the retina.

Furthermore another Mitogen-activated protein kinase (MAPK) cascade, one of the major protein kinase families involved in intracellular signaling has been implicated in the activation of Anti-endothelial cell antibodies (AECA) in the sera of patients with Behcet's disease (BD) and uveitis. AECA of the IgM subtype can play a pathogenic role in induction of vasculitis and inflammatory lesions of BD by directly activating endothelial cells (HDMEC), independent from the help of proinflammatory cytokines such as TNF alpha or IL-1 alpha. These antibodies facilitate the perpetuation of a chronic inflammatory response by attracting lymphocytes to leave the bloodstream and infiltrate the eye. Inhibition of the enzymes of the MAPK cascade pathways stopped the antibody production.

In summary, even though the evidence of the role of small molecule inhibitors in the treatment of uveitis is still largely unexplored, preliminary evidence suggests that small molecule inhibitors may play an important role in the treatment of uveitis in the near future.

Since multiple signaling pathways are known to be involved in all of the diseases discussed, it is very likely that the most effective local treatment for these diseases will be to use multiple compounds that are selective to the disease-specific pathways that cause the inflammation and/or the degeneration. The current treatment paradigm for degenerative eye diseases has been to administer a single compound, usually systemically, even though it has been shown that the separate processes of inflammation and neovascularization occur simultaneously. Targeting multiple pathways, by using combinations of anti-cytokines, anti-chemokines, kinase inhibitors, and other signal modulating agents, delivered locally, will allow the treatment of these eye diseases with superior outcomes and safety, and represent a new approach to the treatment of the leading causes of blindness. Because of the complexity of these diseases, it is not yet clear whether the best treatment option would be a single implanted delivery device that releases multiple compounds or a collection of implanted delivery devices that each releases only a single compound, each of which would allow a physician to tailor the treatment to achieve specific treatment profiles.

Therefore, a heretofore unaddressed need exists in the art to address the aforementioned deficiencies and inadequacies.

### SUMMARY OF THE INVENTION

In one aspect, the present invention relates to an implant for intraocular drug delivery for the treatment of intraocular inflammatory or degenerative diseases. In one embodiment, the implant includes a body portion. The body portion has a first end portion, a second, opposite end portion, an outer surface, an interior surface, and a length L defined between the first end portion end and the second end portion. The body portion defines a cavity with a first opening at the first end portion, and a second, opposite opening at the second end portion. In one embodiment, the body portion has a cross-section of a circle, a square, an oval, or a polygon. The implant further includes a solid material received in the cavity, where the solid material comprises a depot material and an effective amount of at least one therapeutic compound or agent.

The implant may also include a first membrane covering the first opening of the body portion, through which the at least one therapeutic compound or agent is controllably released to the environment of the implant, and a second membrane covering the second opening of the body portion, through which the at least one therapeutic compound or agent is controllably released to the environment of the implant. The first membrane and the second membrane each is made from a biodegradable material.

In one embodiment, the implant is implanted in or around the vitreous or other parts of the posterior chamber of the eye of a living subject so that the cavity of the implant is in fluid communication with the vitreous or other parts of the posterior chamber of the eye through at least one of the first opening and the second, opposite opening. When the implant is implanted in an eye of a living subject, the effective amount of at least one therapeutic compound or agent is released to the environment of the implant through at least one of the first opening and the second, opposite opening over an extended period of time. In one embodiment, the effective amount of at least one therapeutic compound or agent is released to the environment of the implant by diffusion through and dissolution of the depot material that comprises a soluble binder material.

The body portion of the implant, in one embodiment, is made from an inert polymeric material selected from polysulfone, polyetherimide, polyimide, polymethylmethacrylate, siloxanes, other acrylates, polyetheretherketone, copolymers of any of these compounds, and biocompatible implantable polymers.

In another embodiment, the body portion of the implant is made from a biodegradable material such that when the effective amount of at least one therapeutic compound is released to the environment of the implant, the body portion gradually resorbs or degrades in situ. The biodegradable material includes a biodegradable polymeric material selected from modified poly(saccharides), including starch, cellulose, and chitosan, fibrin, fibronectin, gelatin, collagen, collagenoids, tartrates, gellan gum, dextran, maltodextrin, poly(ethylene glycol), poly(propylene oxide), poly(butylene oxide), Pluoronics, modified polyesters, poly(lactic acid), poly(glycolic acid), poly(lactic-co-glycolic acid), modified alginates, carbopol, poly(N-isopropylacrylamide), poly(lysine), triglyceride, polyanhydrides, poly(ortho)esters, poly(epsilon-caprolactone), poly(butylene terephthalate), polycarbonates, triglyceride, copolymers of glutamic acid and leucine, poly(hydroxyalkanoates) of the PHB-PHV class, proteins, polypeptides, proteoglycans, polyelectolytes, and any copolymer or combination of them.

The soluble binder material, in one embodiment, comprises at least one of modified poly(saccharides), including starch, cellulose, and chitosan, sugars and modified sugars, including trehalose, sucrose, sucrose esters, polyalcohols, poly(vinyl alcohol), glycerol, fibrin, fibronectin, gelatin, collagen, collagenoids, tartrates, gellan gum, heparin, carrageenan, pectin, xanthan, dextran, maltodextrin, poly(ethylene glycol), poly(propylene oxide), poly(butylene oxide), Pluoronics, modified alginate hydrogels, carbopol, poly(lysine), proteins, polypeptides, polyelectolytes, proteoglycans, and any copolymer or combination of them.

The at least one therapeutic compound or agent, in one embodiment, comprises at least one biologic immunomodulator or anti-inflammatory agent that specifically or functionally oppose the action of Tumor Necrosis Factor alpha (TNFα); the Interleukines including Interleukine-1, Interleukine-2, Interleukine-4, Interleukine-6, Interleukine-8, Interleukine-12, Interleukine-15, Interleukine-17, and Interleukine-18; Anti-chemokines and anti-metalloproteases that specifically or functionally oppose the action of MCP-1 (9-76), Gro-alpha (8-73), V MIPII, CXCR4, Met-CCL5, Met-RANTES, CCR1, RANTES (CCL5), MIP 1 alpha (CCL3), IP 10 (CXCL10), VEGF, MCP 1-4 (CCL1, CCL8, CCL7, CCL13), CINC, Cognate receptor, GRO, CXCR4, Stromal-derived factor-1, CCR4, CCR5, and CXCR3; Chemokines or synthetic molecules that are structurally or functionally equivalent to Interleukine-10 and Interleukine-12; and Tumor Growth Factors (TGF) and related anti-inflammatory growth factors. Co-stimulatory molecule inhibitor including CTLA4 Ig, anti CD11, anti CD2, fusion protein of LFA3e and IgGFc; inhibitors of nitric oxide (NO) or inducible nitric oxide synthase (iNOS), adhesion molecule inhibitors including alpha4-integrin inhibitor, inhibitors of P selectin or E selectin or ICAM1 or VCAM, alpha-melanocyte stimulating hormone (alpha-MSH), anti HSP 60 or Heme Oxygenase (HO)-1, and heat shock proteins.

The at least one therapeutic compound or agent may also comprise at least one of the following signal pathway modulators or involve in the signaling pathways to reduce or inhibit inflammation and angiogenesis, including NF-kappa B inhibitors such as Pyrrolidine dithiocarbamate (PTDC), Proteasome inhibitor, MG-132, Rolipram, an inhibitor of type 4 phosphodiesterase, CM101, for example; inhibitors of other transcription factors such as activator protein 1 (AP1), activating transcription factor 2 (ATF2), nuclear factor of activated T cells (NF-AT), signal transducer and activator of transcription (STAT), p53, Ets family of transcription factors (Elk-1 and SAP-1), nuclear hormone receptors; small molecule inhibitors that inhibit or block the following intracellular signaling pathways, or regulatory enzymes/kinases, for example: PTEN, PI3 Kinases, P38 MAP Kinase and other MAP Kinases, all stress activated protein kinases (SAPKs), the ERK signaling pathways, the JNK signaling pathways (JNK1, JNK2), all RAS activated pathways, all Rho mediated pathways, and all NIK, MEKK-1, IKK-1, IKK-2 pathways; and other intracellular and extracellular signaling pathways.

In another embodiment, the at least one therapeutic compound or agent comprises any combination of the agents mentioned above.

In an alternative embodiment, the at least one therapeutic compound or agent comprises at least one of antibodies, nanobodies, antibody fragments, signaling pathway inhibitors, transcription factor inhibitors, receptor antagonists, small molecule inhibitors, oligonucleotides, fusion proteins, peptides, protein fragments, allosteric modulators of cell surface receptors such as G-protein coupled receptors (GPCR), cell surface receptor internalization inducers, and GPCR inverse agonists.

In another aspect, the present invention relates to an implant for intraocular drug delivery. In one embodiment, the implant has a body portion having an outer surface and an interior surface, where the interior surface defines a cavity with at least one opening. In one embodiment, the outer surface of the body portion has a geometric shape of a hemisphere. The implant also has an effective amount of at least one therapeutic compound or agent received in the cavity, where when the implant is implanted in the eye of a living subject, the effective amount of at least one therapeutic compound or agent is released to the environment of the implant through the at least one opening over an extended period of time.

The implant further has a soluble binder material, where at least one therapeutic compound or agent is stabilized with the soluble binder material to form a compound that is received in the cavity. The soluble binder material comprises at least one of modified poly(saccharides), including starch, cellulose, and chitosan, sugars and modified sugars, including trehalose, sucrose, sucrose esters, polyalcohols, poly(vinyl alcohol), glycerol, fibrin, fibronectin, gelatin, collagen, collagenoids, tartrates, gellan gum, heparin, carrageenan, pectin, xanthan, dextran, maltodextrin, poly(ethylene glycol), poly(propylene oxide), poly(butylene oxide), Pluoronics, modified alginate hydrogels, carbopol, poly(lysine), proteins, polypeptides, polyelectolytes, proteoglycans, and any copolymer or combination of them.

In one embodiment, the implant may comprises a membrane covering the at least one opening of the body portion, through which the at least one therapeutic compound or agent is controllably released to the environment of the implant, where the membrane is made from a biodegradable material.

The body portion of the implant in one embodiment is made from an inert polymeric material selected from the group of polysulfone, polyetherimide, polyimide, polymethylmethacrylate, siloxanes, other acrylates, polyetheretherketone, copolymers of any of the these compounds, and similar engineered biocompatible implantable polymers.

In another embodiment the body portion is made from a biodegradable material such that when the effective amount of at least one therapeutic compound is released to the environment of the implant, the body portion gradually resorbs or degrades in situ. The biodegradable material comprises a biodegradable polymeric material selected from modified poly(saccharides), including starch, cellulose, and chitosan, fibrin, fibronectin, gelatin, collagen, collagenoids, tartrates, gellan gum, dextran, maltodextrin, poly(ethylene glycol), poly(propylene oxide), poly(butylene oxide), Pluoronics, modified polyesters, poly(lactic actid), poly(glycolic acid), poly(lactic-co-glycolic acid), modified alginates, carbopol, poly(N-isopropylacrylamide), poly(lysine), triglyceride, polyanhydrides, poly(ortho)esters, poly(epsilon-caprolactone), poly(butylene terephthalate), polycarbonates, triglyceride, copolymers of glutamic acid and leucine, poly(hydroxyalkanoates) of the PHB-PHV class, proteins, polypeptides, proteoglycans, polyelectolytes, and any copolymer or combination of them.

In one embodiment, the at least one therapeutic compound or agent comprises at least one immunomodulator or anti-inflammatory agent that specifically or functionally opposes the action of Tumor Necrosis Factor alpha (TNFα); the Interleukines including Interleukine-1, Interleukine-2, Interleukine-4, Interleukine-6, Interleukine-8, Interleukine-12, Interleukine-15, Interleukine-17, and Interleukine-18; Anti-chemokines and anti-metalloproteases that specifically or functionally oppose the action of MCP-1 (9-76), Gro-alpha (8-73), V MIPII, CXCR4, Met-CCL5, Met-RANTES, CCR1, RANTES (CCL5), MIP 1 alpha (CCL3), IP 10 (CXCL10), VEGF, MCP 1-4 (CCL1, CCL8, CCL7, CCL13), CINC, Cognate receptor, GRO, CXCR4, Stromal-derived factor-1, CCR4, CCR5, and CXCR3; Chemokines or synthetic molecules that are structurally or functionally equivalent to Interleukine-10 and Interleukine-12; and Tumor Growth Factors (TGF) and related anti-inflammatory growth factors; co-stimulatory molecule inhibitor including CTLA4 Ig, anti CD11, anti CD2, fusion protein of LFA3e and IgGFc; inhibitors of nitric oxide (NO) or inducible nitric oxide synthase (iNOS); adhesion molecule inhibitors including alpha4-integrin inhibitor; inhibitors of P selectin or E selectin or ICAM1 or VCAM; alpha-melanocyte stimulating hormone (alpha-MSH); anti HSP 60 or Heme Oxygenase (HO)-1; and heat shock proteins.

The at least one therapeutic compound or agent may also comprise at least one of the following signal pathway modulators or involve in the following pathways to reduce or inhibit inflammation and angiogenesis, including NF-kappa B inhibitors such as Pyrrolidine dithiocarbamate (PTDC), Proteasome inhibitor, MG-132, Rolipram, an inhibitor of type 4 phosphodiesterase, CM101, for example; inhibitors of other transcription factors such as activator protein 1 (AP1), activating transcription factor 2 (ATF2), nuclear factor of activated T cells (NF-AT), signal transducer and activator of transcription (STAT), p53, Ets family of transcription factors (Elk-1 and SAP-1), nuclear hormone receptors; small molecule inhibitors that inhibit or block the following intracellular signaling pathways, or regulatory enzymes/kinases, for example: PTEN, PI3 Kinases, P38 MAP Kinase and other MAP Kinases, all stress activated protein kinases (SAPKs), the ERK signaling pathways, the JNK signaling pathways (JNK1, JNK2), all RAS activated pathways, all Rho mediated pathways, and all NIK, MEKK-1, IKK-1, IKK-2 pathways; and other intracellular and extracellular signaling pathways.

In another embodiment, the at least one therapeutic compound or agent comprises at least two therapeutic compounds, at least one of which is an anti-cytokine or anti-chemokine for the treatment of inflammatory diseases by simultaneously and synergistically blocking signal transduction pathways involved in the inflammatory and/or autoimmune disorders related to the eye of a living subject.

In yet another embodiment, the at least one therapeutic compound or agent comprises at least one of antibodies, nanobodies, antibody fragments, signaling pathway inhibitors, transcription factor inhibitors, receptor antagonists, small molecule inhibitors, oligonucleotides, fusion proteins, peptides, protein fragments, interference RNA, allosteric modulators of cell surface receptors such as G-protein coupled receptors (GPCR), cell surface receptor internalization inducers, and GPCR inverse agonists.

In one embodiment, the at least one therapeutic compound or agent is in the form of a plurality of particles, which are releasable to the environment of the implant.

The effective amount of at least one therapeutic compound or agent, in one embodiment, is released to the environment of the implant by diffusion through and dissolution of the soluble binder material.

In one embodiment, when the implant is implanted in the eye of a living subject, the implant is placed in or around the vitreous or other parts of the posterior chamber of the eye of a living subject so that the cavity of the implant is in fluid communication with the vitreous or other parts of the posterior chamber of the eye through the at least one opening.

In yet another aspect, the present invention relates to an eye implant. In one embodiment, the eye implant includes a first material, and a second material containing an effective amount of at least one therapeutic compound or agent, where the first material and the second material are arranged to form a solid, and when the eye implant is implanted in an eye of a living subject, the effective amount of at least one therapeutic compound or agent is releasable to the environment of the implant over an extended period of time. The eye implant may comprise a third material containing an effective amount of at least one therapeutic compound or agent.

In one embodiment, the first material and the second material are formed in a layer structure. In another embodiment, the first material, the second material and the third material are formed in a layer structure. When the eye implant is implanted in the eye of a living subject, materials in different layers are released to the environment of the eye implant at different rates, respectively or one after another.

Alternatively, the first material and the second material are formed in a wafer-like structure. The first material and the second material may be also formed to a solid such that at any given position, the density of the material is substantially one of the densities of the first material and the density of the second material.

In one embodiment, the first material comprises an inert polymeric material selected from the group of polysulfone, polyetherimide, polyimide, polymethylmethacrylate, siloxanes, other acrylates, polyetheretherketone, copolymers of any of the these compounds, and similar engineered biocompatible implantable polymers.

The first material in another embodiment comprises a biodegradable material such that when the effective amount of at least one therapeutic compound or agent is released to the environment of the eye implant, the first material gradually degrades or dissolves in situ. The biodegradable material comprises a biodegradable polymeric material selected from modified poly(saccharides), including starch, cellulose, and chitosan, fibrin, fibronectin, gelatin, collagen, collagenoids, tartrates, gellan gum, dextran, maltodextrin, poly(ethylene glycol), poly(propylene oxide), poly(butylene oxide), Pluoronics, modified polyesters, poly(lactic actid), poly(glycolic acid), poly(lactic-co-glycolic acid), modified alginates, carbopol, poly(N-isopropylacrylamide), poly(lysine), triglyceride, polyanhydrides, poly(ortho)esters, poly(epsilon-caprolactone), poly(butylene terephthalate), polycarbonates, triglyceride, copolymers of glutamic acid and leucine, poly(hydroxyalkanoates) of the PHB-PHV class, proteins, polypeptides, proteoglycans, polyelectolytes, and any copolymer or combination of them.

The second material further comprises a soluble binder material. The at least one therapeutic compound or agent is stabilized with the soluble binder material. The soluble binder material in one embodiment comprises at least one of modified poly(saccharides), including starch, cellulose, and chitosan, sugars and modified sugars, including trehalose, sucrose, sucrose esters, polyalcohols, poly(vinyl alcohol), glycerol, fibrin, fibronectin, gelatin, collagen, collagenoids, tartrates, gellan gum, heparin, carrageenan, pectin, xanthan, dextran, maltodextrin, poly(ethylene glycol), poly(propylene oxide), poly(butylene oxide), Pluoronics, modified alginate hydrogels, carbopol, poly(lysine), proteins, polypeptides, polyclectolytes, proteoglycans, and any copolymer or combination of them.

The effective amount of at least one therapeutic compound or agent is released to the environment of the eye implant by diffusion through and dissolution of the soluble binder material.

In one embodiment, when the eye implant is implanted in the eye of a living subject, the eye implant is placed in or around the vitreous or other parts of the posterior chamber of the eye of a living subject.

In a further aspect, the present invention relates to a method of treating inflammatory and degenerative diseases in or around the eye. In one embodiment, the method includes the step of providing an eye implant having a first material, and a second material containing an effective amount of at least one therapeutic compound or agent, where the first material and the second material are arranged to form a solid. Furthermore, the method includes the step of implanting the eye implant in an eye of a living subject. The effective amount of at least one therapeutic compound is releasable to the environment of the eye implant over an extended period of time. The method also includes the step of leaving the eye implant in the eye.

In one embodiment, the first material comprises an inert polymeric material selected from the group of polysulfone, polyetherimide, polyimide, polymethylmethacrylate, siloxanes, other acrylates, polyetheretherketone, copolymers of any of the these compounds, and similar engineered biocompatible implantable polymers. In another embodiment, the first material comprises a biodegradable material such that when the effective amount of at least one therapeutic compound or agent is released to the environment of the eye implant, the first material gradually degrades or dissolves in situ.

The second material further comprises a soluble binder material, and wherein at least one therapeutic compound or agent is stabilized with the soluble binder material. The effective amount of at least one therapeutic compound or agent is released to the environment of the eye implant by diffusion through and dissolution of the soluble binder material.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment, and wherein:

FIG. 1 shows schematically an implant according to one embodiment of the present invention: (a) a perspective view, and (b) a cross sectional view.

FIG. 2 shows schematically an implant according to another embodiment of the present invention: (a) a perspective view, and (b) a cross sectional view.

FIG. 3 shows schematically an implant according to yet another embodiment of the present invention: (a) a perspective view, and (b) a cross sectional view.

FIG. 4 shows schematically an implant according to an alternative embodiment of the present invention: (a) in a first state, (b) a second state, and (c) a third state.

FIG. 5 shows schematically an implant according to one embodiment of the present invention: (a) a perspective view, and (b) a sectional view.

FIG. 6 shows schematically an implant according to another embodiment of the present invention: (a) a perspective view, (b) a partially cross sectional view, and (c) compounds and/or agents in the implant releasing to the environment.

FIG. 7 shows schematically an implant according to an alternative embodiment of the present invention: (a) a perspective view, and (b) a cross sectional view.

FIG. 8 shows schematically an implant according to a further embodiment of the present invention.

FIG. 9 shows schematically an implant according to yet a further embodiment of the present invention: (a) in a first state, and (b) in a second state.

FIG. 10 shows schematically an implant according to one embodiment of the present invention: (a) a cross sectional view, and (b) compounds and/or agents in the implant.

### DETAILED DESCRIPTION OF THE INVENTION

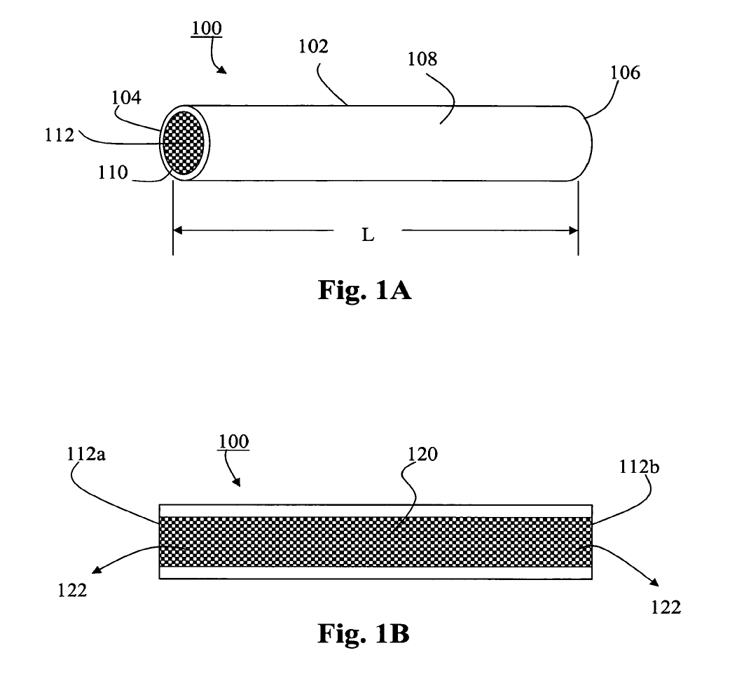

The present invention is more particularly described in the following examples that are intended as illustrative only since numerous modifications and variations therein will be apparent to those skilled in the art. Various embodiments of the invention are now described in detail. Referring to the drawings of FIGS. 1-10, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### DEFINITIONS

The terms used in this specification generally have their ordinary meanings in the art, within the context of the invention, and in the specific context where each term is used.

Certain terms that are used to describe the invention are discussed below, or elsewhere in the specification, to provide additional guidance to the practitioner in describing the apparatus and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification.

Furthermore, subtitles may be used to help a reader of the specification to read through the specification, which the usage of subtitles, however, has no influence on the scope of the invention.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used, the term “uveitis” is referred generally to intraocular inflammations, which account for at least 50 different entities with either infectious or autoimmune origin, Primary uveitis (“idiopathic”) is referred to the intraocular inflammation of unknown cause (roughly 40% of cases seen in tertiary referral centers). Secondary uveitis (all cases with some explanation for the uveitis) accounts for inflammatory ocular conditions that are either associated with a systemic disease (e.g. ankylosing spondylitis or sarcoidosis) of known infectious cause (e.g. toxoplasmosis or CMV-retinitis) or defined as ocular syndromes (e.g. Fuchs uveitis syndrome, Birdshot syndrome or serpiginous choroiditis). Masquerade syndromes, like intraocular lymphoma, are different from primary or secondary uveitis.

The term “compound” is referred to a chemical combination of two or more elements that may have an impact on any living system such as a cell, nerve or tissue. Examples of compounds that may be related to practicing the present invention include those in the following exemplary list:

### Anti-Inflammatory Compounds:

a) Anti-Cytokines
Anti-Tumor Necrosis Factor alpha (TNFα) such as
(1) Etanercept (p75 TNFr fusion protein)
(2) Infliximab (chimeric Anti TNF Mab)
(3) Adalimumab (human Anti TNF Mab)
(4) Onercept (soluble p55 TNFr)
Or other compounds, such as antibodies, nanobodies, antibody fragments, and receptor antagonists.
Anti-Interleukin-1 such as
(1) Anakinra (IL-1 type 1 receptor antagonist)
(2) IL1 Trap (Regeneron, an IL-1 type 1 receptor plus IL-1 fusion protein) or other compounds
Anti-Interleukin-2 such as
(1) Daclizumab or other compounds
Anti-Interleukin-4 such as
(1) Human Anti-IL-4 antibody, E coli derived goat IgG (R&D systems)
(2) Human Anti-IL-4 antibody, E coli derived murine IgG (R&D systems)
Or other compounds
Anti-Interleukin-6 such as
(1) MRA (Chugai Pharmaceuticals/Roche) or other compounds
Anti-Interleukin-8 such as
(1) Anti-EGF-R antibody (C225) or other compounds
Anti-Interleukin-12 such as
(1) Human Anti-IL-12 antibody, E coli derived goat IgG (R&D systems)
(2) Human Anti-IL-12 antibody, E coli derived murine IgG (R&D systems)
Or other compounds
Anti-Interleukin-15 such as
(1) Human Anti-IL-15 antibody, E coli derived goat IgG (R&D systems)
(2) Human Anti-IL-15 antibody, E coli derived murine IgG (R&D systems)
Or other compounds
Anti-Interleukin-17 such as
(1) Human Anti-IL-17 antibody, E coli derived goat IgG (R&D systems)
(2) Human Anti-IL-17 antibody, E coli derived murine IgG (R&D systems)
Or other compounds
Anti-Interleukin-18 such as
(1) Human Anti-IL-18 antibody, E coli derived goat IgG (R&D systems)
(2) Human Anti-IL-18 antibody, E coli derived murine IgG (R&D systems)
Or other compounds

b) Cytokines
Interleukin 10 and 12

c) TGF Beta and Related Anti-Inflammatory Growth Factors

d) Anti-Chemokines/Anti-Metalloproteases
MCP-1 (9-76),
Gro-alpha (8-73),
V MIPII
CXCR4
Met-CCL5
Met-RANTES
oral CCR1 antagonist and others
And all other potential compounds which antagonize the following chemokines and metalloproteases or its receptors:
RANTES (CCL5)
MIP 1 alpha (CCL3)
IP 10 (CXCL10)
VEGF
MCP 1-4 (CCL1, CCL8, CCL7, CCL13)
CINC
Cognate receptor
GRO
CXCR4
Stromal-derived factor-1
CCR4, CCR5, and CXCR3 and others

e) Co Stimulatory Molecule Inhibitors:
CTLA4 Ig
Efalizumab (anti CD11a) binds to unique CD11a chain of LFA1
Alefacept (anti CD2) fusion protein of LFA3e and IgGFc and others

f) Inhibitors of Nitric Oxide (NO) or Inducible Nitric Oxide Synthase (iNOS)

g) Other
Adhesion molecule inhibitors: such as alpha4-integrin inhibitor, inhibitors of P selectin or E selectin, ICAM1, VCAM and others
Alpha-melanocyte stimulating hormone (alpha-MSH)
Anti HSP 60 or Heme oxygenase (HO)-1, heat shock proteins

### Anti-Angiogenic/Anti-Degenerative Compounds:

a) NF-kappa B inhibitors such as
   Pyrrolidine dithiocarbamate (PTDC)
   Proteasome inhibitor, MG-132
   Rolipram, an inhibitor of type 4 phosphodiesterase
   CM101
   And others
b) Inhibitors of other transcription factors such as
   Activator protein 1 (AP1)
   Activating transcription factor 2 (ATF2)
   Nuclear factor of activated T cells (NF-AT)
   Signal transducer and activator of transcription (STAT)
   p53
   Ets family of transcription factors (Elk-1 and SAP-1)
   Nuclear hormone receptors
c) Small molecule inhibitors that inhibit or block the following intracellular signaling pathways, or regulatory enzymes/kinases, for examples:
   PTEN
   PI3 Kinases
   P38 MAP Kinase and other MAP Kinases
   All stress activated protein kinases (SAPKs)
   The ERK signaling pathways
   The JNK signaling pathways (JNK1, JNK2)
   All RAS activated pathways
   All Rho mediated pathways
   NIK, MEKK-1, IKK-1, IKK-2.

Tumor Necrosis Factor alpha (TNFα) plays a pivotal role in most animal models of uveitis. In addition it regulates most cytokines and chemokines and indirectly influences the inflammatory process. Multiple clinical trials have demonstrated that TNF inhibition is beneficial in treating uveitis and other inflammatory eye conditions such as Behcet's disease (BD) [13,16]. Currently available TNF inhibitors include Etanercept (p75 TNFr fusion protein), Infliximab (chimeric Anti TNF Mab), Adalimumab (human Anti TNF Mab), and Onercept (soluble p55 TNFr). Currently applied doses for various autoimmune diseases: Etanercept: 50 mg once a week SQ or 0.8 mg/kg/wk for a child; Adalimumab: 40 mg EOW SQ or app. 1 mg/kg/wk for a child; and Infliximab: 3-10 mg/kg at 0, 2, 6 weeks and then every other month IV. Infliximab has been shown to improve vision in patients with degenerative diseases such as choroidal neovascularization [19], macular edema [20, 23], macular degeneration [21], and branch retinal vein occlusion [22].

Interleukin-1 (IL-1) appears to have a more pivotal role in endotoxin induced uveitis than TNF-alpha, and IL-1 beta is one of the principal mediators of LPS-induced uveitis. IL-1 may act as local amplification signal in pathological processes associated with chronic eye inflammation [10]. IL-1beta causes blood brain barrier (BRB) breakdown by opening tight junctions between RVE cells and possibly by increasing transendothelial vesicular transport. Currently available IL-1 inhibitors include [1] Anakinra (IL-1 type 1 receptor antagonist) and IL1 Trap (Regeneron, an IL-1 type 1 receptor plus IL-1 fusion protein). In addition synthetic IL-1 blockers (CK-138, 139) are effective in treatment of IL-1 alpha induced uveitis in the rat. Currently applied doses for various autoimmune diseases: Anakinra: 100 mg/d SQ or app. 1 mg/kg/d for a child.

IL-2 is initially identified as a T cell growth factor that is produced by T cells following activation by mitogens or antigens. Since then, it has also been shown to stimulate the growth and differentiation of B cells, natural killer (NK) cells, lymphocyte activated killer (LAK) cells, monocytes/macrophages and oligodendrocytes. At the amino acid sequence level, there is approximately 72% similarity between mature porcine and human IL-2 and approximately 80% similarity between rat and mouse IL-2. IL-2 is expressed upon stimulation of T-cells and is a commonly used marker for T-cell activation. The primary, known physiologic effect of IL-2 is to act as a T lymphocyte growth factor. Elevated aqueous and serum levels of IL-2 have been observed in patients with uveitis, especially with acute anterior uveitis and BD [2, 9, 11]. Suppression of serum IL2 levels has been shown to be beneficial in animals and humans with various forms of uveitis [1]. Currently available IL-2 inhibitors include Daclizumab, a monoclonal antibody, that exerts its effect by binding to the alpha subunit (CD25) of the human interleukin (IL)-2 receptor on the surface of activated lymphocytes, thus preventing the binding of IL-2. Currently applied doses for transplant rejection: 1 mg/kg/dose for a total of 5 doses for children and adults.

IL-4 is a pleiotropic cytokine produced by activated T cells, mast cells, and basophiles. It was initially identified as a B cell differentiation factor (BCDF), as well as a B cell stimulatory factor (BSF1). IL-4 has since been shown to have multiple biological effects on hematopoietic and non-hematopoietic cells, including B and T cells, monocytes, macrophages, mast cells, myeloid and erythroid progenitors, fibroblasts, and endothelial cells. Rat, mouse and human IL-4 are species-specific in their activities. IL-4 can induce the production of IFN-gamma and other inflammatory cytokines under certain conditions. IL-4 can exert a dose-dependent differential effect on the induction of immune responses and on autoimmunity. IL4 is an important cytokine in the regulation of IL6 and perhaps other cytokine production by endothelium in vivo. IL-4 secreting cells are significantly increased in active BD. Active and in remission BD patients have increased serum levels of IL-4. PBMC from patients with BD produced higher levels of IL-4. In addition IL-4 plays an important role in the late phase of EAU. Similarly, treatment with IL-4 significantly decreased the development of uveitis from 68% to 30.4% in rats with HSP induced uveitis. Furthermore there are significantly elevated IL-4 levels in aqueous humors of patients with complicated cataracts. Anti-Interleukin-4 (IL-4) includes human anti-IL-4 antibody, E coli derived goat IgG (R&D systems), human anti-IL-4 antibody, E coli derived murine IgG (R&D systems), or other compounds.

IL-6 is also known as interferon-b2, 26-kDa protein, B cell stimulatory factor-2 (BSF-2), hybridoma/plasmacytoma growth factor, hepatocyte stimulating factor, cytotoxic T cell differentiation factor, and macrophage-granulocyte inducing factor 2A (MGI-2A). IL-6 is a multi-functional protein that plays important roles in host defense, acute phase reactions, immune responses, and hematopoiesis [4, 8, 14, 18]. IL-6 is expressed by a variety of normal and transformed cells including T cells, B cells, monocytes/macrophages, fibroblasts, hepatocytes, keratinocytes, astrocytes, vascular endothelial cells, and various tumor cells. It plays an important role as an inflammatory mediator in VKH [15]. In addition especially IL-6 levels increase significantly following laser photocoagulation and IL-6 is one of the dominant contributing factors in the occurrence of postoperative inflammation. Currently applied doses for arthritis: 8 mg/kg/dose for children and adults. Anti-Interleukin-6 (IL-6) includes MRA (Chugai Pharmaceuticals) or other compounds. IL-6 is one of several elevated pro-inflammatory signaling molecules found in both macular degeneration and branch vein occlusion [21, 22].

IL-8 is also referred to as neutrophil chemotactic factor (NCF), neutrophil activating protein (NAP), monocyte-derived neutrophil chemotactic factor (MDNCF), T cell chemotactic factor (TCF), granulocyte chemotactic protein (GCP) and leukocyte adhesion inhibitor (LAI). Many cell types, including monocyte/macrophages, T cells, neutrophils, fibroblasts, endothelial cells, keratinocytes, hepatocytes, chondrocytes, and various tumor cell lines, can produce IL-8 in response to a wide variety of pro-inflammatory stimuli such as exposure to IL-1, TNF, LPS, and viruses. IL-8 is a member of the CXC subfamily of chemokines. IL-8 plays a role in the progression of intraocular inflammation, and granulocytes are thought to be a possible source of IL-8 in endophthalmitis [7]. IL-8 contributes to the chemotactic signal for the recruitment of leukocytes in EIU. Anti-IL-8 antibody treatment partially blocks EIU in rabbits. IL-8 is one of the dominant contributing factors in the occurrence of postoperative inflammation. IL-8 mediated mechanisms are responsible for ocular lesions in BD and there is a close relationship between the cell-associated IL-8 and the disease activity. Anti-Interleukin-8 (IL-8) has anti-EGF-R antibody (C225) or other compounds.

IL-12 is also known as natural killer cell stimulatory factor (NKSF) or cytotoxic lymphocyte maturation factor (CLMF), and it is a hetero-dimeric pleiotropic cytokine made up of a 40 kDa (p40) subunit and a 35 kDa (p35) subunit. The IL-12 p40 subunit is shared by IL-23, another heterodimeric cytokine that has biological activities similar to, as well as distinct from, IL-12. IL-12 is produced by macrophages and B cells and has been shown to have multiple effects on T cells and natural killer (NK) cells. While mouse IL-12 is active on both human and mouse cells, human IL-12 is not active on mouse cells. IL-12 is a cytokine that facilitates cytolytic T-cell responses, enhances the lytic activity of NK cells and induces the secretion of interferon-gamma by both T and NK cells. IL-12 plays a pivotal role in the initiation and maintenance of the intraocular inflammation. IL-12 has an inhibitory effect on endotoxin-induced inflammation in the eye suggesting that IL-12 can have an immunoregulatory function in some forms of inflammatory eye disease. High levels of IL-12 in the vitreous and/or aqueous humor in patients with uveitis of non-neoplastic etiology have been observed [5, 6]. Serum IL-12 levels are associated with a general clinical improvement during treatment. In addition IL-12 plays a substantial part in the pathogenesis of BD and there is a correlation of IL-12 plasma levels with disease activity, so that anti-IL-12 or pro-IL-12 or IL-12 itself may be of use depending on specific clinical symptoms. Anti-Interleukin-12 (IL-12) includes human anti-IL-12 antibody, E coli derived goat IgG (R&D systems), human anti-IL-12 antibody, E coli derived murine IgG (R&D systems), or other compounds.

IL-15 shares many biological properties with IL-2, including T, B and natural killer cell-stimulatory activities. Human IL-15 shares approximately 97% and 73% sequence identity with simian and mouse IL-15, respectively. Both human and simian IL-15 are active on mouse cells. IL-15 mRNA is expressed by a wide variety of cells and tissues and is most abundantly expressed by adherent peripheral blood mononuclear cells, fibroblasts and epithelial cells. IL-15 is a novel cytokine that induces T cell proliferation, B cell maturation, natural killer cell cytotoxicity, and may have a pivotal role in the pathogenesis of inflammatory disease, acting upstream from tumour necrosis factor alpha (TNF alpha). IL-15 is elevated in RA patients, especially in those with long-term disease and is involved in the perpetuation of RA synovitis. IL-15 and interleukin 18 (IL18) are cytokines produced principally by macrophages during innate immune response and subsequently profoundly influence adaptive immunity. In addition this cytokine plays an important role in the biology of pathologic scar formation and is involved in the regulation of apoptosis. Its exact role in uveitis is still unclear. Anti-Interleukin-15 (IL-15) includes humans anti-IL-15 antibody, E coli derived goat IgG (R&D systems), humans anti-IL-15 antibody, E coli derived murine IgG (R&D systems), or other compounds.

IL-17 is also known as CTLA-8, is a T cell-expressed pleiotropic cytokine that exhibits a high degree of homology to a protein encoded by the ORF13 gene of herpes virus Saimiri. Both recombinant and natural IL-17 have been shown to exist as disulfide linked homo-dimers. At the amino acid level, human IL-17 shows 72% and 63% sequence identity with herpes virus and rat IL-17, respectively. The IL-17 family comprises at least six members, including IL-17, IL-17B, IL-17C, IL-17D, IL-17E (IL-25) and IL-17F. All IL-17 family members share a set of spatially conserved cysteine residues, which suggest that IL-17 family members may be related to the cysteine knot superfamily. IL-17 upregulates the expression of several pro-inflammatory cytokines and it modulates the immune response during viral infections. IL17 may act as a potent upstream mediator of cartilage collagen breakdown in inflammatory joint diseases but its exact role in uveitis is still unclear. Active BD was characterized by a higher increase of IL-17 compared to remission BD. Anti-Interleukin-17 (IL-17) includes human anti-IL-17 antibody, E coli derived goat IgG (R&D systems), human anti-IL-17 antibody, E coli derived murine IgG (R&D systems), or other compounds.

IL-18 is also known as interferon-gamma-inducing factor (IGIF) and IL-1g, and it is a cytokine which shares biologic activities with IL-12 and structural similarities with the IL-1 family of proteins. Porcine IL-18 cDNA encodes a precursor molecule (pro-IL-18) that shares 77% sequence identity with human pro-IL-18. Pro-IL-18 lacks a hydrophobic signal peptide but contains a leader sequence that is analogous to the IL-1b pro-domain. IL-18 is expressed in the epithelial cells in iris, ciliary body, and retina in the eyes, but its role in the eye remains undetermined. IL-18 up-regulation is a feature of BD and suggests that IL-18 may contribute to the local inflammatory response. Active BD was characterized by a higher increase of IL-18 and IFN-gamma, compared to remission BD. Anti-Interleukin-18 (IL-18) includes human anti-IL-18 antibody, E coli derived goat IgG (R&D systems), human anti-IL-18 antibody, E coli derived murine IgG (R&D systems), or other compounds.

Tumor growth factor beta two, TGFβ-2, is reduced below normal in ocular inflammation such as Fuch's heterochromic cyclitis [12]. The etiology is unknown, but restoration of normal levels in the vitreous could help to reduce severity as the compound is known to be neuroprotective in some animals. Interferon gamma, IFNγ, may be one of the mediators for induced expression of HLA antigens on iris cells which may play a role in the pathogenesis of anterior uveitis and iritis [17].

Anti-Chemokines and Anti-Metalloproteases (ACM): Anti-chemokines and anti-metalloproteases which specifically or functionally oppose the action of MCP-1 (9-76), Gro-alpha (8-73), V MIPII, CXCR4, Met-CCL5, Met-RANTES, CCR1, RANTES (CCL5), MIP 1 alpha (CCL3), IP 10 (CXCL10), VEGF, MCP 1-4 (CCL1, CCL8, CCL7, CCL13), CINC, Cognate receptor, GRO, CXCR4, Stromal-derived factor-1, CCR4, CCR5, CXCR3 and the like.

Chemokines [chemoattractant cytokines and Matrix Metallo Proteinases (MMPs)] comprises a complex super family of at least 40-50 low molecular weight proteins (usually between 6-14 KD). They have varying cellular targets and biological responses. High levels of MMPs are found in patients with chronic uveitis and contribute to the damage often seen in these eyes. Since MMPs are capable of releasing proinflammatory cytokines bound to components of the extracellular matrix, and facilitate the secretion of active TNF-alpha by cleavage of the membrane bound form, it is conceivable that MMPs contribute to the chronicity of some uveitis cases. The amounts of IL-1beta, IL-12 and IL-1ra correlate with levels of MMP-2 and MMP-9. CXC chemokine GRO is essential for neutrophil infiltration in LPS-induced uveitis in rabbits. Most of GRO production is mediated by TNF alpha and IL-1. GRO and IL-8 act in concert to mediate neutrophil infiltration.

Some representative examples of chemokines include: RANTES (CCL5), MIP 1 alpha (CCL3), IP 10 (CXCL10), VEGF, MCP 1-4 (CCL1, CCL8, CCL7, CCL13), CINC, Cognate receptor, GRO, CXCR4, and Stromal-derived factor-1.

Chemokine antagonists are available in the form of MCP-1(9-76), Gro-alpha(8-73), vMIPII, CXCR4, Met-CCL5, Met-RANTES and have been shown to be beneficial in rat models of arthritis and glomerulonephritis as well as murine models of atherosclerosis, spinal cord injury, and tumor.

Cytokines (CK): IL-10 is an anti-inflammatory or inflammation modulating cytokine which has been found to reduce the effects of many of the cytokines listed above [3]. IL-12 is usually pro-inflammatory but there are some indications that it also has a regulatory role in the suppression of specific immune responses. Treatment using molecules which are structurally or functionally equivalent to Interleukine-10 and Interleukine-12 may help to reduce inflammation in some disease states.

Other signal pathway modulators: Other signal pathway molecules are well known to those versed in the art, the following list is not exclusive or complete but contains those factors whose modulation could prove useful in the control of inflammation and/or degeneration of ocular tissue: co-stimulatory molecule inhibitor including CTLA4 Ig, anti CD11, anti CD2, fusion protein of LFA3e and IgGFc; inhibitors of nitric oxide (NO) or inducible nitric oxide synthase (iNOS); adhesion molecule inhibitors including alpha4-integrin inhibitor, inhibitors of P selectin or E selectin or ICAM1 or VCAM, alpha-melanocyte stimulating hormone (alpha-MSH), anti HSP 60 or Heme Oxygenase (HO)-1, heat shock proteins; NF-kappa B inhibitors such as Pyrrolidine dithiocarbamate (PTDC), Proteasome inhibitor, MG-132, Rolipram, an inhibitor of type 4 phosphodiesterase, CM101, for example; inhibitors of other transcription factors such as activator protein 1 (AP1), activating transcription factor 2 (ATF2), nuclear factor of activated T cells (NF-AT), signal transducer and activator of transcription (STAT), p53, Ets family of transcription factors (Elk-1 and SAP-1), nuclear hormone receptors; small molecule inhibitors that inhibit or block the following intracellular signaling pathways, or regulatory enzymes/kinases, for example: PTEN, PI3 Kinases, P38 MAP Kinase and other MAP Kinases, all stress activated protein kinases (SAPKs), the ERK signaling pathways, the JNK signaling pathways (JNK1, JNK2), all RAS activated pathways, all Rho mediated pathways, and all NIK, MEKK-1, IKK-1, IKK-2 pathways; and other intracellular and extracellular signaling pathways.

The term “agent” is broadly defined as anything that may have an impact on any living system such as a cell, nerve or tissue. For examples, the agent can be a chemical agent. The agent can also be a biological agent. The agent may comprise at least one known component. The agent can also be a physical agent. Other examples of agent include biological warfare agents, chemical warfare agents, bacterial agents, viral agents, other pathogenic microorganisms, emerging or engineered threat agents, acutely toxic industrial chemicals (TICS), toxic industrial materials (TIMS) and the like. Preferably, biological or pharmacological agents are employed to practice the present invention. Examples of agent types that may be related to practicing the present invention include antibodies, nanobodies, antibody fragments, signaling pathway inhibitors, transcription factor inhibitors, receptor antagonists, small molecule inhibitors, oligonucleotides, fusion proteins, peptides, protein fragments, allosteric modulators of cell surface receptors such as G-protein coupled receptors (GPCR), cell surface receptor internalization inducers, and GPCR inverse agonists.

The term “inert polymeric material” is referred to a biocompatible non-degrading polymer that includes but is not limited to one of polysulfone, polyetherimide, polyimide, polymethylmethacrylate, siloxanes, other acrylates, polyetheretherketone, copolymers of any of the these compounds, and similar engineered biocompatible implantable polymers.

The term “biodegradable material” is referred to a material that may be selected from modified poly(saccharides), including starch, cellulose, and chitosan, fibrin, fibronectin, gelatin, collagen, collagenoids, tartrates, gellan gum, dextran, maltodextrin, poly(ethylene glycol), poly(propylene oxide), poly(butylene oxide), Pluoronics, modified polyesters, poly(lactic actid), poly(glycolic acid), poly(lactic-co-glycolic acid), modified alginates, carbopol, poly(N-isopropylacrylamide), poly(lysine), triglyceride, polyanhydrides, poly(ortho)esters, poly(epsilon-caprolactone), poly(butylene terephthalate), polycarbonates, triglyceride, copolymers of glutamic acid and leucine, poly(hydroxyalkanoates) of the PHB-PHV class, proteins, polypeptides, proteoglycans, polyelectolytes, and any copolymer or combination of them, in addition to other materials well known to those versed in the art and which appear in the scientific and technical literature.

The term “soluble binder” is referred to a material that is selected from the following list, which is not a complete enumeration of the many choices available to those skilled in the art: modified poly(saccharides), including starch, cellulose, and chitosan, sugars and modified sugars, including trehalose, sucrose, sucrose esters, polyalcohols, poly(vinyl alcohol), glycerol, fibrin, fibronectin, gelatin, collagen, collagenoids, tartrates, gellan gum, heparin, carrageenan, pectin, xanthan, dextran, maltodextrin, poly(ethylene glycol), poly(propylene oxide), poly(butylene oxide), Pluoronics, modified alginate hydrogels, carbopol, poly(lysine), proteins, polypeptides, polyelectolytes, proteoglycans, and any copolymer or combination of them.

The term “depot material” is referred to a material that includes at least one of a biodegradable material, a soluble binder or any combinations of them.

### DESCRIPTION OF THE PREFERRED EMBODIMENTS

Among other things, the present invention relates to the treatment of chronic disorders of the eye that may and can cause long-term damage including vision loss or blindness.

Various treatment options have been developed for patients who are affected by these disorders. In case of the inflammatory eye diseases, for examples, patients are treated with a combination of immunosuppressive medications in addition to topical steroid eye drops. This has three major disadvantages: it may leave the patients vulnerable to infections; it could cause damage to their inner organs, especially liver and kidney; and it may cause cataracts and increase intraocular pressure (glaucoma) in the eye. In case of the degenerative vasculopathies, moreover, existing treatments are not generally effectual.

The present invention provides a different approach and offers a viable and superior treatment solution for inflammatory and/or degenerative eye diseases. By delivering signal pathway modulating drugs directly into the eye in situ through the device(s) and method(s) according to several embodiments of the present invention, systemic side effects can be avoided and precise treatment of the disease at the site is enabled.

Thus, among other things, the present invention allows delivery of compounds or agents, such as monoclonal antibodies or kinase inhibitors, directly into an eye of a living subject such as a patient or a animal, which may allow one to dramatically reduce chronic eye diseases by modulating the signal pathways to suppress inflammation without suppression of the immune system and allow dramatic reduction in the formation of new blood vessels thus preventing bleeding and retinal detachment.

Without intent to limit the scope of the invention, various embodiments of the present invention are described below.

The present invention discloses an implant having a first material, and a second material containing an effective amount of at least one therapeutic compound or agent. When the implant is implanted in an eye of a living subject, the effective amount of at least one therapeutic compound or agent is releasable to the environment of the implant over an extended period of time for the treatment of intraocular inflammatory and/or degenerative eye diseases therein.

Referring to FIG. 1, an implant 100 is shown according to one embodiment of the present invention. In this embodiment, the implant 100 includes a body portion 102. The body portion 102 has a first end portion 104, a second, opposite end portion 106, an outer surface 108, an interior surface 110, and a length L defined between the first end portion end 104 and the second end portion 106. The body portion 102 defines a cavity 112 with a first opening 112a at the first end portion 104, and a second, opposite opening 112b at the second end portion 106. In this embodiment, the body portion 102 has a cross-section of a circle. The body portion 102 can also has other cross-section shapes such as a square, an oval, or a polygon.

The implant 100 further includes a solid material 120 received in the cavity 112. The solid material 120 includes a depot material and an effective amount of at least one therapeutic compound or agent 122, where the effective amount of at least one therapeutic compound or agent is released to the environment of the implant 100 by diffusion through and dissolution of the depot material. The depot material has a soluble binder material.

The implant may also include a first membrane covering the first opening 112a of the body portion 102, through which the at least one therapeutic compound or agent is controllably released to the environment of the implant 100, and a second membrane covering the second opening 112b of the body portion 102, through which the at least one therapeutic compound or agent is controllably released to the environment of the implant 100. The first membrane and the second membrane each is made from a biodegradable material.

The body portion 102 of the implant 100, in one embodiment, is made from an inert polymeric material selected from polysulfone, polyetherimide, polyimide, polymethylmethacrylate, siloxanes, other acrylates, polyetheretherketone, copolymers of any of these compounds, and biocompatible implantable polymers. For this embodiment, the body portion 102 still exists and substantially keeps its physical form when and after the effective amount of at least one therapeutic compound is released to the environment of the implant 100.

In another embodiment, the body portion 102 of the implant 100 is made from a biodegradable material such that when the effective amount of at least one therapeutic compound is released to the environment of the implant 100, the body portion 102 gradually resorbs or degrades in situ. In other words, for this embodiment, the body portion 102 gradually disappears and no longer exists in its physical form when and after the effective amount of at least one therapeutic compound is released to the environment of the implant 100. The biodegradable material includes a biodegradable polymeric material selected from modified poly(saccharides), fibrin, fibronectin, gelatin, collagen, collagenoids, tartrates, gellan gum, dextran, maltodextrin, poly(ethylene glycol), poly(propylene oxide), poly(butylene oxide), Pluoronics, modified polyesters, poly(lactic actid), poly(glycolic acid), poly(lactic-co-glycolic acid), modified alginates, carbopol, poly(N-isopropylacrylamide), poly(lysine), triglyceride, polyanhydrides, poly(ortho)esters, poly(epsilon-caprolactone), poly(butylene terephthalate), polycarbonates, triglyceride, copolymers of glutamic acid and leucine, poly(hydroxyalkanoates) of the PHB-PHV class, proteins, polypeptides, proteoglycans, polyelectolytes, and any copolymer or combination of them. The modified poly(saccharides) includes starch, cellulose, and chitosan.

The soluble binder material comprises at least one of modified poly(saccharides), sugars and modified sugars, including trehalose, sucrose, sucrose esters, polyalcohols, poly(vinyl alcohol), glycerol, fibrin, fibronectin, gelatin, collagen, collagenoids, tartrates, gellan gum, heparin, carrageenan, pectin, xanthan, dextran, maltodextrin, poly(ethylene glycol), poly(propylene oxide), poly(butylene oxide), Pluoronics, modified alginate hydrogels, carbopol, poly(lysine), proteins, polypeptides, polyelectolytes, proteoglycans, and any copolymer or combination of them. The modified poly(saccharides) includes starch, cellulose, and chitosan.

The at least one therapeutic compound or agent, in one embodiment, includes at least one of the following signal pathway modulators or involves in the following signaling pathways that specifically or functionally oppose the action of Tumor Necrosis Factor alpha (TNFα); the Interleukines including Interleukine-1, Interleukine-2, Interleukine-4, Interleukine-6, Interleukine-8, Interleukine-12, Interleukine-15, Interleukine-17, and Interleukine-18; Anti-chemokines and anti-metalloproteases that specifically or functionally oppose the action of MCP-1 (9-76), Gro-alpha (8-73), V MIPII, CXCR4, Met-CCL5, Met-RANTES, CCR1, RANTES (CCL5), MIP 1 alpha (CCL3), IP 10 (CXCL10), VEGF, MCP 1-4 (CCL1, CCL8, CCL7, CCL13), CINC, Cognate receptor, GRO, CXCR4, Stromal-derived factor-1, CCR4, CCR5, and CXCR3; Chemokines or synthetic molecules that are structurally or functionally equivalent to Interleukine-10 and Interleukine-12; and Tumor Growth Factors (TGF) and related anti-inflammatory growth factors, co-stimulatory molecule inhibitor including CTLA4 Ig, anti CD11, anti CD2, fusion protein of LFA3e and IgGFc; inhibitors of nitric oxide (NO) or inducible nitric oxide synthase (iNOS), adhesion molecule inhibitors including alpha4-integrin inhibitor, inhibitors of P selectin or E selectin or ICAM1 or VCAM, alpha-melanocyte stimulating hormone (alpha-MSH), anti HSP 60 or Heme Oxygenase (HO)-1, heat shock proteins; NF-kappa B inhibitors such as Pyrrolidine dithiocarbamate (PTDC), Proteasome inhibitor, MG-132, Rolipram, an inhibitor of type 4 phosphodiesterase, CM101, for example; inhibitors of other transcription factors such as activator protein 1 (AP1), activating transcription factor 2 (ATF2), nuclear factor of activated T cells (NF-AT), signal transducer and activator of transcription (STAT), p53, Ets family of transcription factors (Elk-1 and SAP-1), nuclear hormone receptors; small molecule inhibitors that inhibit or block the following intracellular signaling pathways, or regulatory enzymes/kinases, for example: PTEN, PI3 Kinases, P38 MAP Kinase and other MAP Kinases, all stress activated protein kinases (SAPKs), the ERK signaling pathways, the JNK signaling pathways (JNK1, JNK2), all RAS activated pathways, all Rho mediated pathways, and all related NIK, MEKK-1, IKK-1, IKK-2 pathways; and other intracellular and extracellular signaling pathways.

In one embodiment, the implant 100 is implanted in or around the vitreous or other parts of the posterior chamber of the eye of a living subject so that the cavity 112 of the implant 100 is in fluid communication with the vitreous or other parts of the posterior chamber of the eye through at least one of the first opening 112a and the second, opposite opening 112b.

Other implantation sites for place the implant 100 includes the Canal of Petit, the retrozonular space, the uvea, the choroid of the posterior chamber of the eye, the ciliary body, the zonules, pars plana, the ciliary process, the ciliary muscles, the trabecular meshwork, within the sclera or the conjunctiva or at the boundary of the sclera and the conjunctiva, within the anterior chamber of the eye in the anterior chamber in the anatomical angle, Schlemm's Canal, in the cornea at or near the limbus.

When the implant 100 is implanted in an eye of a living subject, the effective amount of at least one therapeutic compound or agent is released to the environment of the implant 100 through at least one of the first opening 112a and the second, opposite opening 112b over an extended period of time, by diffusion through and dissolution of the soluble binder. The releasing rate of the at least one therapeutic compound or agent, for example, 1×10⁴ U per day, is controllable by varying the interior diameter of the cavity 112 of the implant 100, the density of the at least one therapeutic compound or agent, and the binder dissolution rate. The total amount of the at least one therapeutic compound or agent delivered is controllable by adjusting the length of the body portion 102 of the implant 100. The implant 100 may be left in the eye, removed, or may degrade in situ.

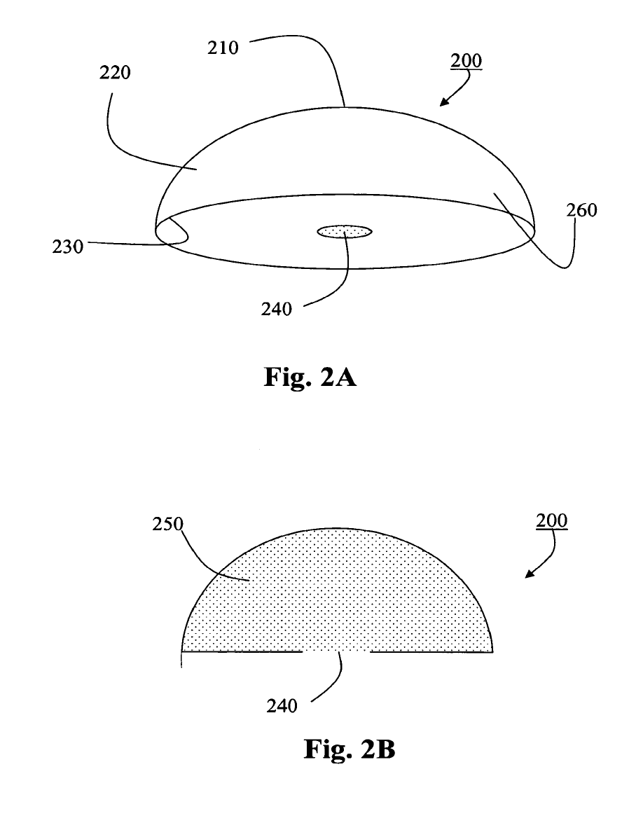

Referring to FIG. 2, another embodiment of an implant 200 of present invention is shown. The implant 200 has a body portion 210 containing a depot material. The body portion 210 has an outer surface 220 and an interior surface 230, where the interior surface 230 defines a cavity 260 with at least one opening 240. In one embodiment, the outer surface 220 of the body portion 210 has a geometric shape of a hemisphere. The outer surface 220 of the body portion 210 can take other geometric shapes. The implant 200 also has an effective amount of at least one therapeutic compound or agent received in the cavity 260. The at least one therapeutic compound or agent is stabilized with the depot material to form a compound 250 that is received in the cavity 260. When the implant 200 is implanted in the eye of a living subject, the effective amount of at least one therapeutic compound or agent is released to the environment of the implant 200 through the at least one opening 240 over an extended period of time.

Optionally, the implant 200 includes a membrane for covering the at least one opening 240 of the body portion 210, through which the at least one therapeutic compound or agent is controllably released to the environment of the implant 200. The membrane can be made from a biodegradable material.

The body portion 210 of the implant in one embodiment can be made from a non-biodegradable material including an inert polymeric material.

Preferably, the hemisphere implant 200 is formed with a biodegradable gel material such as alginate, in which the at least one therapeutic compound or agent (active agent) have been dispersed. The hemisphere implant 200 is covered with a coating that is impermeable to the active agent. The opening 240 in the coating is located near the center of the flat side of the hemisphere implant 200. The active agent, such as Etanercept, an anti-TNFα compound, MCP-1(9-76), or a chemokine antagonist, is released from the opening 240 by diffusion through of the biodegradable material. The rate and total amount of the active agent release is controlled by varying the size of the opening 240, the size of the implant 200, the density of the active agent, and diffusion coefficient of the alginate. After the conclusion of the treatment, (for example, 90 days) the entire implant 200 including coating gradually resorbs or degrades in situ.

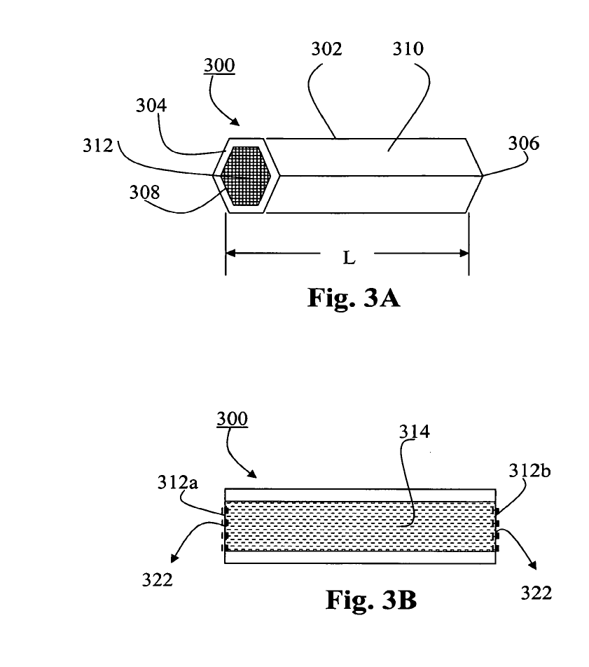

FIG. 3 shows an alternative embodiment of an implant 300 of the present invention. In the embodiment, the implant 300 is formed in the form of a biocompatible polyimide tube 302 having a first end 304, an opposite, second end 306, an interior surface 308 and an exterior surface 310. The interior surface 308 defines a cavity 312 therein. The tube 302 has a cross-section of polygon. The tube 302 may have other types of cross-section or be formed of some other biocompatible material. The cavity 312 of the tube 302 is filled with an active agent, such as Adalimumab, an anti-TNFα antibody and an anti-IL-1 or anti IL-6, compound in an appropriate stabilizing solution 314. The first and second ends 304 and 306 of the tube 302 are sealed with membranes 312a and 312b, respectively, which control the release of the active agent 322 into the surrounding tissue at therapeutic levels for an extended duration, for example, 2 months. The implant 300 may be left in the eye, removed, or may resorb in situ by using degrading materials instead of non-degrading materials.

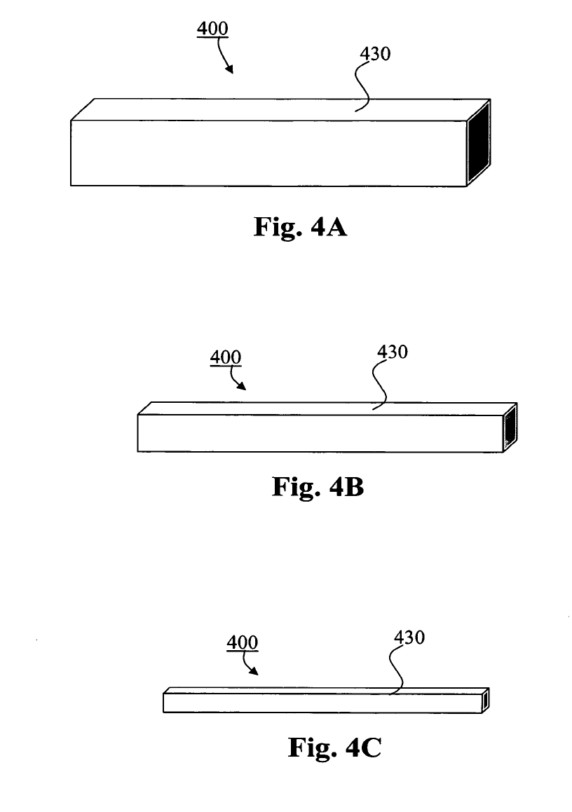

FIG. 4 shows another embodiment of an implant 400 of the present invention. In the embodiment, the implant 400 is formed in the form of a solid, multisided prism 430 with a biodegradable material, such as a polyanhydride, and active agents, for example, monoclonal antibodies. The active agents are dispersed and stabilized within the solid, multisided prism 430. The active agents of the implant 400 are released by diffusion through and degradation of the prism 430 over time. As the treatment proceeds over time, the implant 400 is gently degraded so that the size of the implant 400 is reduced, as shown in FIGS. 4A-4C. For example, FIG. 4A represents the initial size of the implant 400 (in a first state), while FIG. 4B represents the size of the implant 400 at a later time (in a second state), and FIG. 4C represents the size of the implant 400 at a time that is later than the time of FIG. 4B (in a third state). In one embodiment, the rate and total amount of the active agent release is controllable by varying the size of the implant 400, the density of the active agents, and degradation rate of the biodegradable material, individually or in combination.

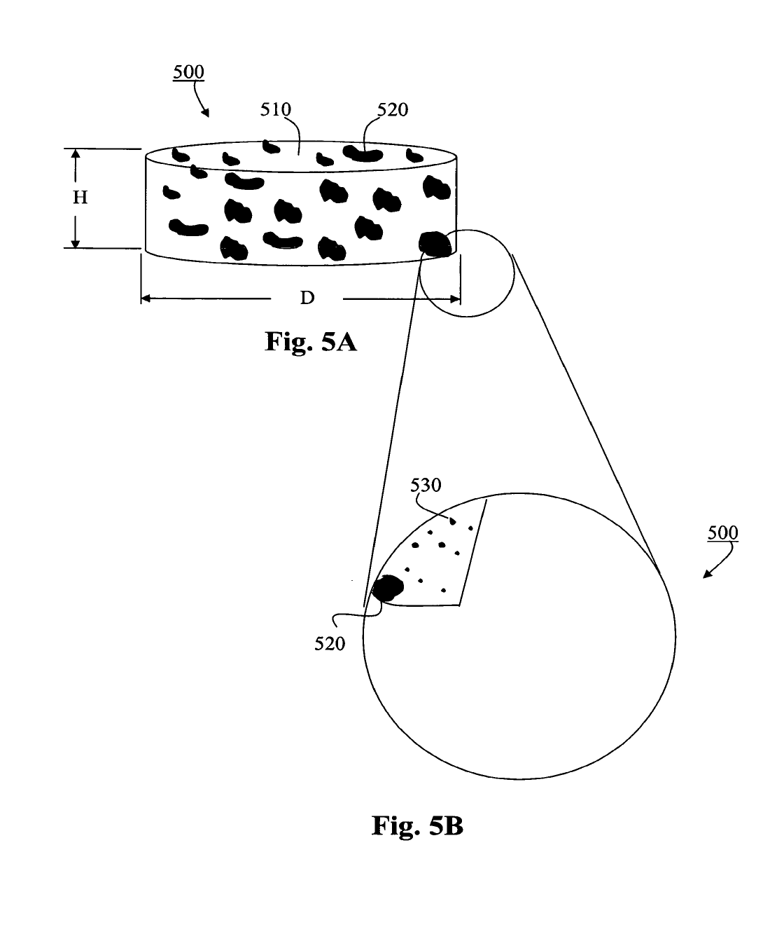

Referring to FIG. 5, an implant 500 is shown according to one embodiment of the present invention. The implant 500 is formed in the form of a cylindrical porous wafer 510 with a biodegradable material, such as poly(lactic-co-glycolic) acid, with a number of collections 520 of active agents 530 dispersed and stabilized within the cylindrical porous wafer 510. The cylindrical porous wafer 510 has a height, H, and a diameter, D. The active agents 530, which include antagonists to TNFα, IL2, and IL4 in a ratio of 350:20:1, are released by diffusion through and degradation of the implant 500. The rate and total amount of the active agent release is controlled by varying the porosity, the size of the implant 500 by having different H and/or D, the density of the active agents, and the degradation rate of the biodegradable material. After implanted, the implant 500 is gradually degraded and eventually dispersed in situ.

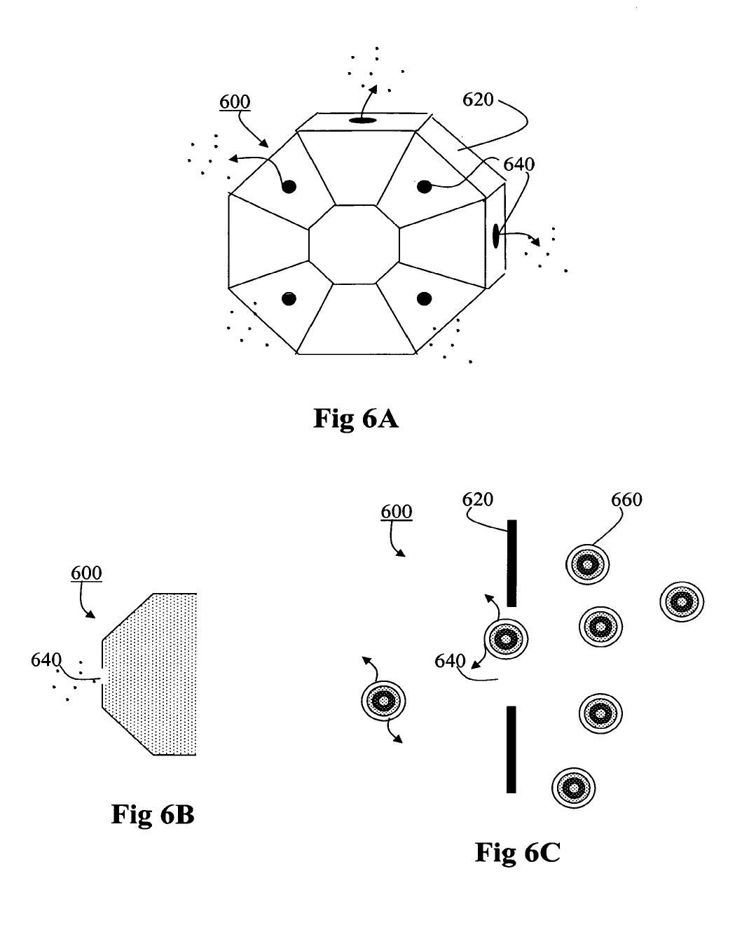

FIG. 6 shows another embodiment of an implant 600 of the present invention. The implant 600 is formed in a hollow multifaceted polyhedron 620 with a biodegradable material, for example, a modified chitosan. The implant 600 has a number of openings 640 formed on surfaces of the hollow multifaceted polyhedron 620. Active agents, e.g., RNA aptamers, are encapsulated in vacuoles 660 of poly(L)lysine and filled in the hollow multifaceted polyhedron 620. After the implant 600 is implanted in a pre-selected implantation site of an eye of a living subject such as a patient or a lab animal, the active agents are released from the interior of the hollow multifaceted polyhedron 620 through the number of openings 640. Following release of the active agents from the vacuoles 660, the implant 600 is gradually degraded and eventually resorbed in situ.

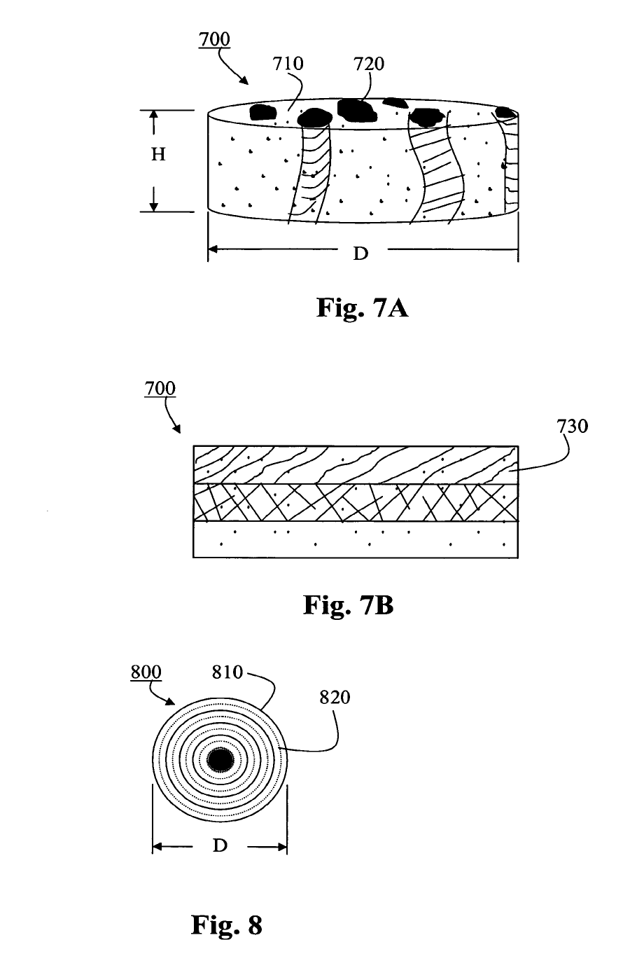

Referring to FIG. 7, an alternative embodiment of an implant 700 is shown. In this embodiment, the implant 700 includes active agents, for example, synthetic antibody fragments, contained by a combination of materials, where each material has a different release profile. For example, the agents are dispersed within a porous biodegradable poly(ortho)ester 710, which releases them over a 6 month period. The pores are filled with agents dispersed in gelatin 720, which releases them over, for example, a 2 week period. In an alternative embodiment, the agents are dispersed in layers of different materials 730 which dissolve at different rates, allowing stepwise control of the release rates as each layer dissolves. The layers can be dissolved one after another, or respectively at same or different rates.

Referring to FIG. 8, an implant 800 is shown according to one embodiment of the present invention. In this embodiment, the implant 800 includes active agents, such as peptides, entrapped in a layer-by-layer structure using compounds of controlled permeability and/or degradation in alternate layers of, for example, polyelectrolytes with opposite charges 810 and 820, like carboxymethylcellulose and protamine sulfate. When the implant 800 is implanted in an implantation site, materials in different layers are released to the environment of the implant 800 at different rates, respectively or one after another.

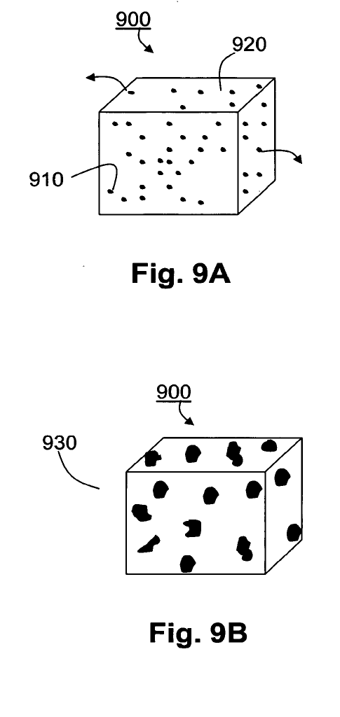

FIG. 9 shows an implant 900 including active agents that are stabilized in layer-by-layer coated particles 910 of pure compound(s) or compound(s) in a depot material, which are entrapped in a degradable matrix 920, such as starch carbonate. The particles 910 degrade and release the active agents at a faster rate than the matrix degrades, leaving behind a sponge-like structure 930 that completely resorbs after the duration of the treatment.

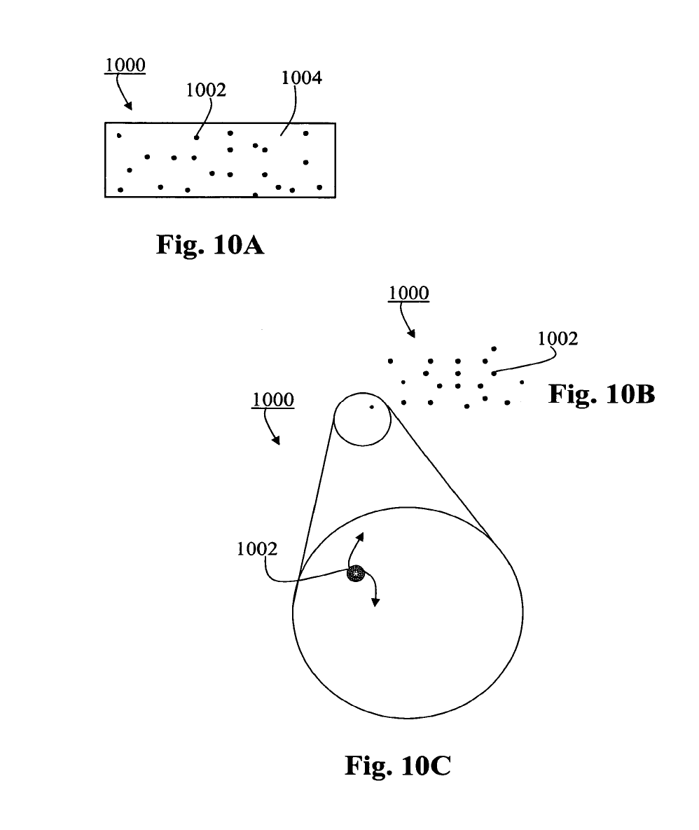

FIG. 10 shows another embodiment of an implant 1000 of the present invention. The implant 1000 comprises active agents that are stabilized in layer-by-layer coated particles 1002 of pure compound(s) or compound(s) in a depot material. The active agents are entrapped in a degradable matrix 1004, such as a starch carbonate. The matrix 1004 degrades and releases the particles 1002, which then begin to release the active agents at a rate depending on both the particle depot material and the coating type and thickness.

Another aspect of the present invention provides a method of treating inflammatory and degenerative diseases in or around the eye. In one embodiment, the method includes the step of providing an eye implant having a first material, and a second material containing an effective amount of at least one therapeutic compound or agent, where the first material and the second material are arranged to form a solid; and when the eye implant is implanted in the eye of a living subject, the effective amount of at least one therapeutic compound or agent is releasable to the environment of the implant over an extended period of time. Furthermore, the method includes the step of implanting the eye implant in an eye of a living subject. The effective amount of at least one therapeutic compound is releasable to the environment of the eye implant over an extended period of time. The method also includes the step of leaving the eye implant in the eye.

The first material includes an inert polymeric material or a biodegradable material such that when the effective amount of at least one therapeutic compound or agent is released to the environment of the eye implant, the first material gradually degrades or dissolves in situ.

The second material further includes a soluble binder material with which the at least one therapeutic compound or agent is stabilized. The effective amount of at least one therapeutic compound or agent is released to the environment of the eye implant by diffusion through and dissolution of the soluble binder material.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

### REFERENCES

1. Murray P I. Clay C D. Mappin C. Salmon M. Molecular analysis of resolving immune responses in uveitis. Clinical & Experimental Immunology. 117 (3):455-61, 1999 September.
2. Lacomba M S. Martin C M. Chamond R R. Galera J M. Omar M. Estevez E C. Aqueous and serum interferon gamma, interleukin (IL) 2, IL-4, and IL-10 in patients with uveitis. Archives of Opthalmology. 118 (6):768-72, 2000 June.
3. Calder V L. Shaer B. Muhaya M. Mclauchlan M. Pearson R V. Jolly G. Towler H M. Lightman S. Increased CD4+ expression and decreased IL-10 in the anterior chamber in idiopathic uveitis. Investigative Opthalmology & Visual Science. 40 (9):2019-24, 1999 August.
4. de Boer J H. van Haren M A. de Vries-Knoppert W A. Baarsma G S. de Jong P V. Postema F J. Rademakers A J. Kijlstra A. Analysis of IL-6 levels in human vitreous fluid obtained from uveitis patients, patients with proliferative intraocular disorders and eye bank eyes. [Journal Article] Current Eye Research. 11 Suppl: 181-6, 1992.
5. Muhaya M. Calder V. Towler H M. Shaer B. McLauchlan M. Lightman S. Characterization of T cells and cytokines in the aqueous humour (AH) in patients with Fuchs' heterochromic cyclitis (FHC) and idiopathic anterior uveitis (IAU). [Journal Article] Clinical & Experimental Immunology. 111 (1):123-8, 1998 January.
6. el-Shabrawi Y. Livir-Rallatos C. Christen W. Baltatzis S. Foster C S. High levels of interleukin-12 in the aqueous humor and vitreous of patients with uveitis. [Journal Article] Opthalmology. 105 (9):1659-63, 1998 September.
7. de Boer J H. Hack C E. Verhoeven A J. Baarsma G S. de Jong P T. Rademakers A J. de Vries-Knoppert W A. Rothova A. Kijlstra A. Chemoattractant and neutrophil degranulation activities related to interleukin-8 in vitreous fluid in uveitis and vitreoretinal disorders. [Journal Article] Investigative Opthalmology & Visual Science. 34 (12):3376-85, 1993 November.
8. Perez V L. Papaliodis G N. Chu D. Anzaar F. Christen W. Foster C S. Elevated levels of interleukin 6 in the vitreous fluid of patients with pars planitis and posterior uveitis: the Massachusetts eye & ear experience and review of previous studies. [Review] [20 refs] [Journal Article. Review. Review, Tutorial] Ocular Immunology & Inflammation. 12 (3):193-201, 2004 September.
9. Muhaya M., Calder V L., Towler H., Jolly G., Mclauchlan M., Lightman S. Characterization of phenotype and cytokine profiles of T cell lines derived from the vitreous humor in ocular inflammation in man. Clinical and Experimental Immunology. 116 (3): 410-414, June 1999.
10. Franks W A. Limb G A. Stanford M R. Ogilvie J. Wolstencroft R A. Chignell A H. Dumonde D C. Cytokines in human intraocular inflammation. Current Eye Research. 11 Suppl: 187-91, 1992.
11. Hooks J J. Chan C C. Detrick B. Identification of the lymphokines, interferon-gamma and interleukin-2, in inflammatory eye diseases. Investigative Opthalmology & Visual Science. 29 (9):1444-51, 1988 September.
12. de Boer J H. Limpens J. Orengo-Nania S. de Jong P T. La Heij E. Kijlstra A. Low mature TGF-beta 2 levels in aqueous humor during uveitis. [Journal Article] Investigative Opthalmology & Visual Science. 35 (10):3702-10, 1994 September.
13. Perez-Guijo V. Santos-Lacomba M. Sanchez-Hernandez M. Castro-Villegas Mdel C. Gallardo-Galera J M. Collantes-Estevez E. Tumour necrosis factor-alpha levels in aqueous humour and serum from patients with uveitis: the involvement of HLA-B27. Current Medical Research & Opinion. 20 (2):155-7, 2004.
14. Murray P I. Hoekzema R. van Haren M A. de Hon F D. Kijlstra A. Aqueous humor interleukin-6 levels in uveitis. Investigative Opthalmology & Visual Science. 31 (5):917-20, 1990 May.
15. Norose K. Yano A. Wang X C. Tokushima T. Umihira J. Seki A. Nohara M. Segawa K. Dominance of activated T cells and interleukin-6 in aqueous humor in Vogt-Koyanagi-Harada disease. Investigative Opthalmology & Visual Science. 35 (1):33-9, 1994 January.
16. Lacomba M S., Martin C M., Gallardo Galera J M, Gomez Vidal M A, Esetevez E C, Chamond R R, Omar M. Aqueous humor and serum tumor necrosis factor-alpha in clinical uveitis. Opthalmic Res 33:251-255, 2001.
17. Abi-Hanna D. McCluskey P. Wakefield D. HLA antigens in the iris and aqueous humor gamma interferon levels in anterior uveitis. [Journal Article] Investigative Opthalmology & Visual Science. 30 (5):990-4, 1989 May.
18. Feys J. Emond J P. Salvanet-Bouccara A. Dublanchet A. [Interleukin-6 and other cytokines in the aqueous humor in uveitis and endophthalmitis]. [French] Journal Francais d Opthalmologie. 17 (11):634-9, 1994.
19. Markomichelakis N N, Theodossiadis P G, Pantelia E, Papaefthimiou S, Theodossiadis G P, Sfikakis P P. Infliximab for chronic cystoid macular edema associated with uveitis. Am J Opthalmol. 2004 October; 138 (4):648-650.
20. Sfikakis P P, Markomichelakis N, Theodossiadis G P, Grigoropoulos V, Katsilambros N, Theodossiadis P G. Regression of sight-threatening macular edema in type 2 diabetes following treatment with the anti-tumor necrosis factor monoclonal antibody infliximab. Diabetes Care. 2005 February; 28 (2):445-447.
21. Markomichelakis N N, Theodossiadis P G, Pantelia E, Papaefthimiou S, Theodossiadis G P, Sfikakis P P. Infliximab for chronic cystoid macular edema associated with uveitis. Am J Opthalmol. 2004 October; 138 (4):648-650.
22. Seddon J M, George S, Rosner B, Rifai N. Progression of age-related macular degeneration: prospective assessment of C-reactive protein, interleukin 6, and other cardiovascular biomarkers. Arch Opthalmol. 2005 June; 123 (6):774-782.
23. Noma H, Funatsu H, Yamasaki M, Tsukamoto H, Mimura T, Sone T, Jian K, Sakamoto I, Nakano K, Yamashita H, Minamoto A, Mishima H K. Pathogenesis of macular edema with branch retinal vein occlusion and intraocular levels of vascular endothelial growth factor and interleukin-6. Am J Opthalmol. 2005 August; 140 (2):256-261.

1-61. (canceled)

62. A method of treating an inflammatory or degenerative disease in or around the eye, comprising the steps of:
a. providing to the eye an eye implant, the eye implant comprising:
i. an effective amount of at least one therapeutic agent, the at least one therapeutic agent comprising at least one anti-TNFα antibody, nanobody, antibody fragment, or combination thereof; anti-interleukine-1 antibody, nanobody, antibody fragment, or combination thereof; anti-interleukine-6 antibody, nanobody, antibody fragment, or combination thereof; anti-interleukine-12 antibody, nanobody, antibody fragment, or combination thereof; or a combination thereof; and
ii. a coating or encapsulating material that coats or encapsulates the at least one therapeutic agent and stabilizes the at least one therapeutic agent;
b. implanting the eye implant in an eye of a living subject, wherein the effective amount of at least one therapeutic agent is releasable to the environment of the eye implant over an extended period of time.

63. The method of claim 62, wherein the flammatory or degenerative disease in or around the eye is uveitis.

64. The method of claim 63, wherein the uveitis is primary uveitis.

65. The method claim 62, wherein the eye implant further comprises a biodegradable or biocompatible material, the coated or encapsulated therapeutic agent being entrapped in the biodegradable or biocompatible material.

66. The method of claim 65, wherein the biodegradable or biocompatible material comprises an inert polymeric material selected from the group of polysulfone, polyetherimide, polyimide, polymethylmethacrylate, siloxanes, polyacrylates, polyetheretherketone, and copolymers thereof.

67. The method of claim 65, wherein the biodegradable material comprises a biodegradable polymeric material selected from modified poly(saccharides), starch, cellulose, chitosan, fibrin, fibronectin, gelatin, collagen, collagenoids, tartrates, gellan gum, dextran, maltodextrin, poly(ethylene glycol), poly(propylene oxide), poly(butylene oxide), Pluoronics, modified polyesters, poly(lactic actid), poly(glycolic acid), poly(lactic-co-glycolic acid), modified alginates, carbopol, poly(N-isopropylacrylamide), poly(lysine), triglyceride, polyanhydrides, poly(ortho)esters, poly(epsilon-caprolactone), poly(butylene terephthalate), polycarbonates, triglyceride, copolymers of glutamic acid and leucine, poly(hydroxyalkanoates) of the PHB-PHV class, proteins, polypeptides, proteoglycans, polyelectolytes, and copolymers and combinations thereof.

68. The method of claim 62, wherein the coating or encapsulating material comprises a polyelectrolyte, a proteoglycan, or both.

69. The method of claim 62, wherein the coating or encapsulating material comprises a proteoglycan.

70. The method of claim 62, wherein the coating or encapsulating material coats the at least one therapeutic agent.

71. The method of claim 62, wherein the at least one therapeutic agent further comprises Interleukine-10, Interleukine-12, or both.

72. The method of claim 62, wherein the second material encapsulates or coats particles of the at least one therapeutic agent.

73. The method of claim 72, wherein particles of the at least one therapeutic agent are coated or encapsulated with a layer-by-layer structure.

74. The method of claim 62, further comprising the step of leaving the eye implant in the eye.
